(19) United States
(12) Patent Application Publication
(10) Pub. No.: US 2024/0023981 A1
(43) Pub. Date: Jan. 25, 2024

(54) THROMBECTOMY DEVICE AND METHOD OF USE THEREOF

(71) Applicant: TAIWAN BIOMATERIAL CO., LTD., Zhubei City, Hsinchu County (TW)

(72) Inventors: Chun-Jen LIAO, Zhubei City (TW); Wen-Hsiang CHANG, Zhubei City (TW); Jia-Yu CHANG, Zhubei City (TW); Wen-Hsi WANG, Zhubei City (TW)

(73) Assignee: TAIWAN BIOMATERIAL CO., LTD., Zhubei City, Hsinchu County (TW)

(21) Appl. No.: 17/768,928

(22) PCT Filed: Oct. 15, 2020
(86) PCT No.: PCT/CN2020/121155

§ 371 Date: Apr. 14, 2022

Related U.S. Application Data
(60) Provisional application No. 62/915,306, filed on Oct. 15, 2019.

(51) Int. Cl. A61B 17/22

(52) U.S. Cl. CPC A61B 17/22031; A61B 2217/005; A61B 2017/00862

(57) ABSTRACT

A thrombectomy device (100) comprising an aspiration pump (10), a catheter (20) and a valve (30) is provided. The aspiration pump (10) is for providing an negative pressure continuously or by interval. The catheter (20) having a distal end (23), mid portion (22) and a proximal end (21) and defining a longitudinal axis. The valve (30) connects between the aspiration pump (10) and the proximal end (21) of the catheter (20) or connects to the catheter (20). Wherein, the catheter (20) comprises at least one elastic area (24), which is compressed along the longitudinal axis in response to application of the negative pressure and expanded along the longitudinal axis in response to relieve of the negative pressure.

### CROSS-REFERENCE TO RELATED APPLICATIONS

The disclosure claims priority to U.S. Provisional Application Ser. No. 62/915,306 filed on, Oct. 15, 2019, the entirety of which is hereby incorporated by reference herein for all purposes.

### FIELD OF THE INVENTION

The disclosure relates in general to a thrombectomy device, and more particularly to method of removing thrombus by using it.

### BACKGROUND OF THE INVENTION

Thrombosis, a main reason of Ischemic stroke, is caused by clot due to abnormal coagulation or debris that blocks the flow of blood in the vessel, which then causes hypoxia of adjacent tissues. Treatment of thrombosis focuses on removal of thrombus. Main stream treatment relies on thrombolytic agents; however, the efficacy of the thrombolytic agents varies patient by patient. Interventional treatment, as known as thrombectomy, is an alternative option to remove thrombus in situ. Briefly, traditional interventional treatment introduces a device into the vessel to remove the thrombus mechanically. However, from time to time, the device may break the thrombus into pieces, and larger pieces may cause thrombosis in other places of the vessel.

Another interventional treatment is related to the usage of pump. In a nutshell, a catheter connected to a pump (or a syringe) is directed into vessel. After the distal end of the catheter arrives the position of the thrombus, the pump will remove the thrombus by sucking it into the catheter. However, if the size of thrombus is too big, it would be hard to be moved, tending to adhere to the wall of the vessel and may block the catheter. Enhancing the negative pressure of catheter would not solve this predicament.

In light of the foregoing, the field continuously needs a better solution to remove large thrombus inside patient body.

### SUMMARY

The disclosure is directed to a thrombectomy device comprising an aspiration pump, a catheter and a valve. The aspiration pump is for providing a negative pressure continuously or by interval. The catheter has a distal end, mid portion and a proximal end and defining a longitudinal axis. The valve is connecting between the aspiration pump and the proximal end of the catheter or connecting to the catheter. Wherein, the catheter further comprises at least one elastic area, which is compressed along the longitudinal axis in response to application of the negative pressure and expanded along the longitudinal axis in response to relieve of the negative pressure.

Preferably, the distal end of the catheter is the elastic area.

Preferably, the proximal end of the catheter is the elastic area.

Preferably, the mid portion of the catheter is the elastic area.

Preferably, the entire catheter is the elastic area.

Preferably, the elastic area is made of a flexible material, and the flexible material includes NiTi alloy and elastic polymer such as polyimide, PU, TPU, silicone, rubber.

Preferably, a spring is disposed in the elastic area of the catheter.

Preferably, the elastic area of the catheter has an accordion fold structure.

According to another embodiment of the present disclosure, a method of removing thrombus using the above thrombectomy device is provided. The method comprising following steps: contacting the distal end of the catheter to a thrombus; turning on the aspiration pump; alternately opening and closing the value to change the length of the elastic area; and moving or breaking the thrombus with an elastic force generated by the compressed/released elastic area.

### BRIEF DESCRIPTION OF THE DRAWINGS

FIGS. 1A to 1C illustrate working examples of the thrombectomy device of the present disclosure.

FIG. 2 shows another embodiment of value.

FIGS. 3A to 3B. show the operation of thrombectomy device according to one embodiment of the present disclosure. FIG. 3A shows the deformation of elastic area 24 in the vessel 40; FIG. 3B omits the vessel and thrombus in FIG. 3A to make the length changing of the elastic area clear.

FIGS. 4-6 show the operation of thrombectomy device according to other embodiments of the present disclosure. The elastic area in FIG. 4 is located in the mid portion of the catheter. In FIG. 5, the elastic area of the catheter has an accordion fold structure. In FIG. 6, the elastic area is solely made of flexible material.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

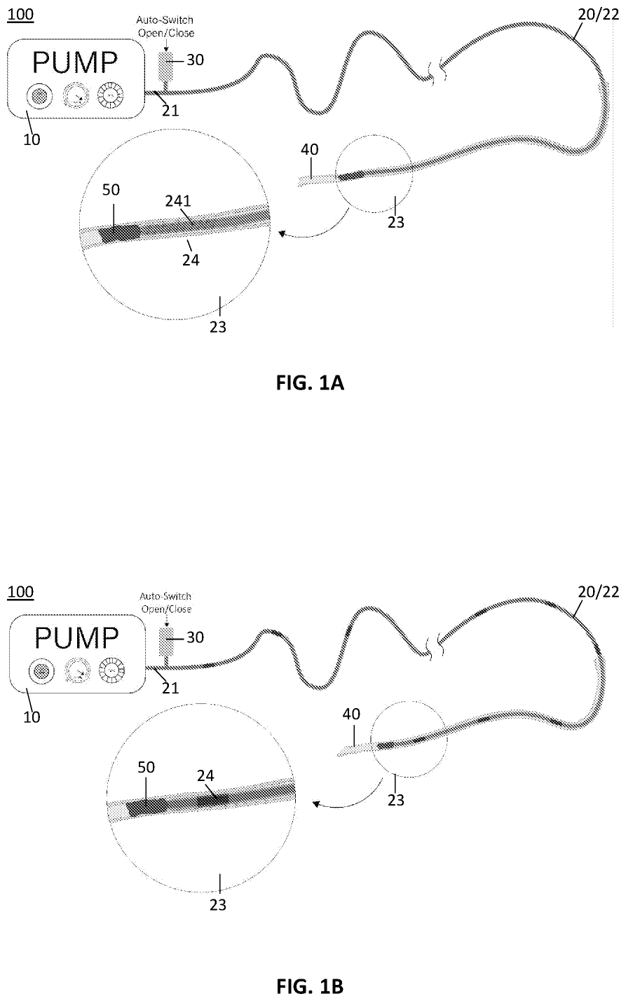

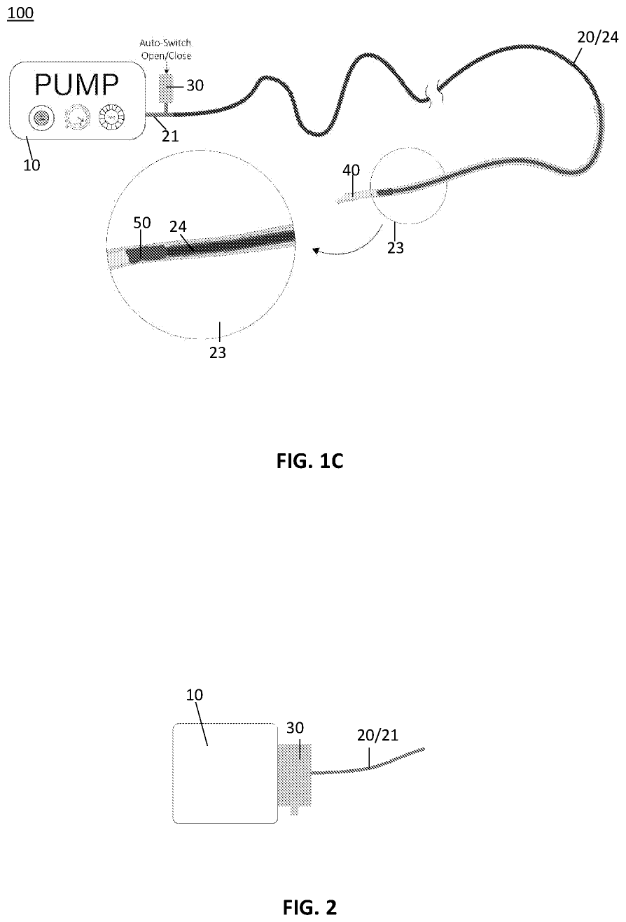

Referring to FIGS. 1A to 1C, which illustrate working examples of the thrombectomy device of the present disclosure. The thrombectomy device 100 comprises an aspiration pump 10, a catheter 20 and a valve 30.

The Pump 10 is an aspiration pump (vacuum pump), which is connecting to the catheter 20 and generates the negative pressure inside the catheter 20 in order to suck, move or break the thrombus 50 in vessel 40.

The catheter 20 comprises a proximal end 21, a mid portion 22 and a distal end 23. The proximal end 21 is positioned outside the body of the subject in need of thrombectomy. In FIG. 1, the proximal end 21 is directly connecting to the aspiration pump 10. However, in another embodiment as shown in FIG. 2, the proximal end 21 is connecting to the pump 10 via the valve 30.

Back to FIGS. 1A to 1C, the distal end 23, on the other hand, is to be introduced into vessel 40 and eventually be positioned close to or contact with the thrombus 50 to be removed. The mid portion 22 is the portion between the proximal end 21 and the distal end 23. The catheter 20 further comprises an elastic area 24.

Referring to FIG. 1A, the elastic area 24 is located in the distal end 23 of the catheter 20. However, the position of the elastic area 24 is not limited in the distal end 23, and could be adjusted on demand. For example, the elastic area 24 could be located in the mid portion 22 and/or the proximal end 21 of the catheter 20. Further, the catheter 20 may comprise a plurality of elastic area 24.

In the embodiment of FIG. 1B, the catheter 20 comprises multiple elastic area 24 (dark region), and these elastic area 24 are located in the proximal end 21, mid portion 22 and distal end 23. In another embodiment of FIG. 1C, the entire catheter 20 is elastic area 24.

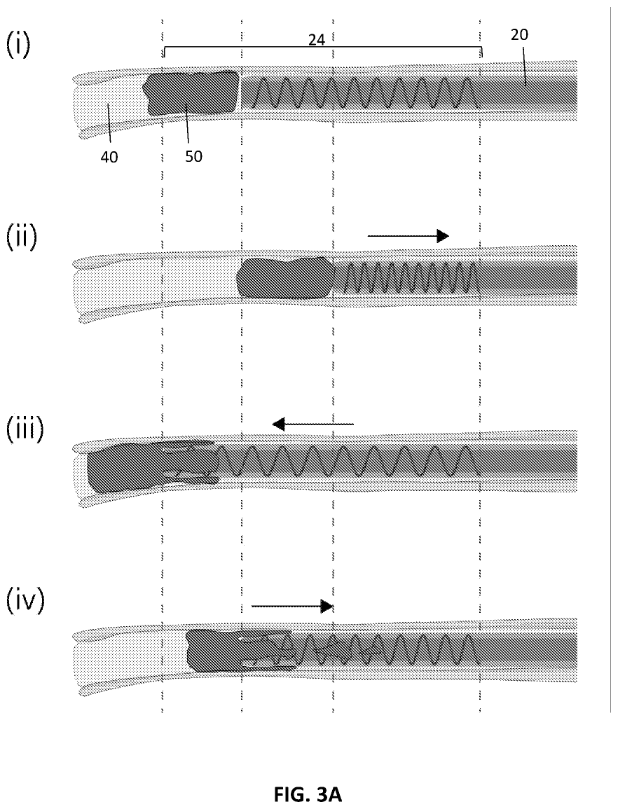

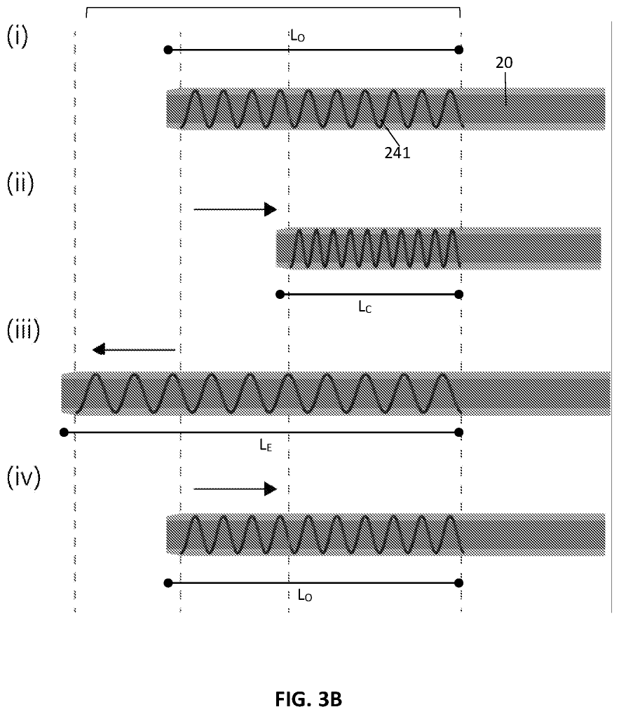

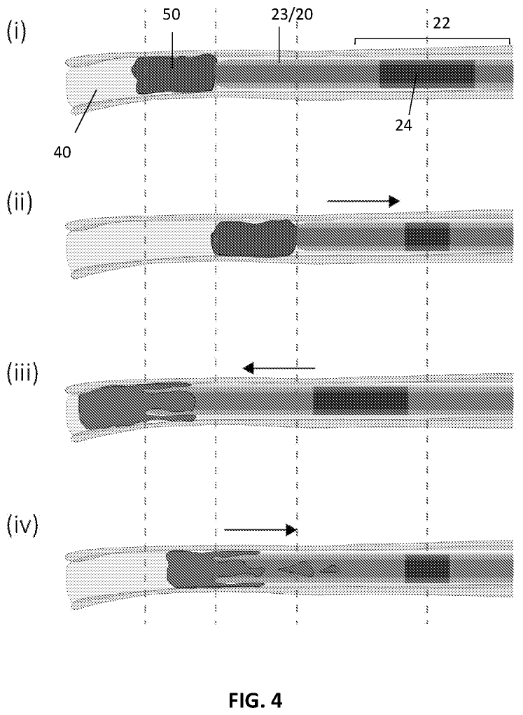

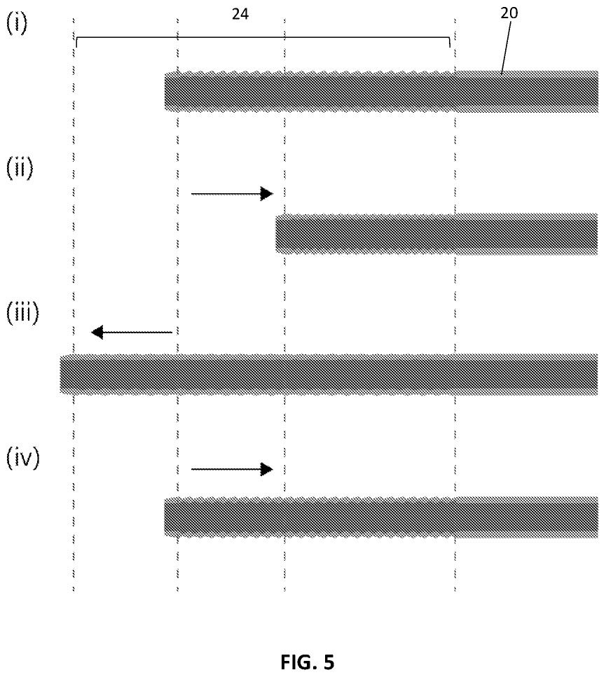

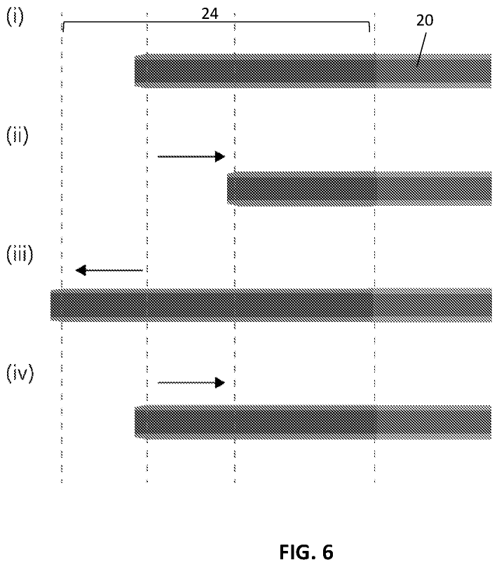

The elastic area 24, as its name indicated, is elastic and could be deformed by the negative pressure applied by the pump 10 (compressed or expanded along the longitudinal axis), thereby changing the length of the elastic area 24. In order to be “elastic”, the elastic area 24 may be made of different material or has different structure from the rest of catheter 20. FIGS. 3-6 show different embodiments of the elastic area 24 and how they operate (described in detail later). In FIGS. 3A and 3B, the elastic area 24 is a spring 241 covered with a flexible material. In FIG. 4, the elastic area 24 is located in the mid portion 22 of the catheter 20. In FIG. 5, the elastic area 24 of the catheter 20 has an accordion fold structure. In FIG. 6, the elastic area 24 is solely made of flexible material, such as flexible polymer or superelasticity alloys. The deformation of elastic area 24 generates an elastic force, which could be used for moving and/or breaking the thrombus.

The location of the elastic area 24 will affect the elastic force generated by the deformation of elastic area 24. For example, the closer the elastic area 24 between the thrombus 50 is, the stronger the force that the thrombus 50 is suffered.

The number/length of the elastic area 24 also affects the elastic force generated by the deformation of elastic area 24. In practice, the curved blood vessel and its branches may interfere the deformation (compress/extend) of elastic area 24. Adding more elastic area 24 or increasing the total length of the elastic area 24 could reduce this interference. FIGS. 1B and 1C show the examples of different elastic area 20 arrangements. In FIG. 1B, the catheter 20 comprises multiple elastic area 24 (dark region); and in FIG. 1C, the entire catheter 20 is elastic area 24. These different arrangements could reduce the interference of environment (i.e., blood vessel condition) and lead enough elastic force to break/move the thrombus.

The valve 30 is used to control or limit the pressure in the catheter 20. When the aspiration pump 10 turning on, it applies negative pressure to the catheter 20 and sucks thrombus into the catheter and/or compresses the elastic area 24. The valve 30 allows the outside fluid (includes but not limit to air flows, liquid, etc.) into to the catheter 20 from an auxiliary passage, and thus relieves the negative pressure. Referring to FIG. 1A, the valve 30 only connects to the catheter, and has a vent to let outside air refills into the catheter 20. In another embodiment (FIG. 2), the valve 30 is a three-way valve, which is disposed between the pump 10 and the catheter 20, and also has a vent to let outside fluid refills into the catheter 20. By alternately opening and closing the valve 30, the user could control the pressure inside the catheter 20 and the length of the elastic area 24. The present disclosure uses the deformation of the elastic area to move and/or break large thrombus in the vessel.

Operation of the Thrombectomy Device

Referring to FIGS. 3A and 3B. FIG. 3A shows the deformation of elastic area 24 in the vessel 40; FIG. 3B omits the vessel 40 and thrombus 50, only keep the elastic area 24 to make the length changing clear.

The deformation of elastic area 24 could be divided to 4 steps (i)-(iv):
(i): Move the distal end 23 (also elastic area 24 in this embodiment) to contacted with (or being close to) the thrombus 50 (FIG. 3A). In this step, the pump is off and the elastic area 24 has its original length LO (FIG. 3B).
(ii): Turn on the aspiration pump, then the thrombus 50 blocked the opening of the elastic area 24. The negative pressure inside the catheter 20 deforms the elastic area 24 (FIG. 3A) along the longitudinal axis. The length of the compressed elastic area 24 is LC, which is shorter than LO (FIG. 3B). The compressed spring 241 stores elastic potential energy and pulls the thrombus 50. The arrow in FIG. 3B shows the direction of force
(iii): Open the valve to recover the pressure inside the catheter 20 and the length of elastic area 24. The elastic potential energy stored in the spring 241 releases and transforms to kinetic energy of elastic area 24. The expanded elastic area 24 hits and pushes the thrombus 50, moves and/or breaks the thrombus 50 to smaller pieces (FIG. 3A). The length of the expanded elastic area 24 is LE, which is longer than LO (FIG. 3B);
(iv): After expansion, the elastic area 24 recovers to its original length LO.

By repeating above steps (i)-(iv) (it could be achieved by alternately opening and closing the valve), the deformation of elastic area 24 (spring 241) could be seen as an oscillation or simple harmonic motion. This movement could push, pull and/or break the large thrombus into smaller pieces/clots, which could be sucked into the catheter, so as to avoid or remove blockage to the vessel 40.

FIG. 4 shows the deformation of elastic area 24 according to another embodiment of the present disclosure. In this case, the elastic area 24 is located in the mid portion 22 of the catheter 20. Like the spring structure of FIG. 3A, the negative pressure applied by the pump would change the length of the elastic area 24. The operation process is the same as the structure of FIGS. 3A and 3B (above section), so no repeat here.

FIG. 5 shows the deformation of elastic area 24 according to another embodiment of the present disclosure (the vessel and thrombus are omitted). In this case, the elastic area 24 of the catheter 20 is accordion fold (also known as Zig-Zag fold or fan fold). Like the spring structure of FIG. 3B, the negative pressure applied by the pump would change the length of the elastic area 24. The operation process is the same as the structure of FIGS. 3A and 3B, so no repeat here.

FIG. 6 shows the deformation of elastic area 24 according to yet another embodiment of the present disclosure (the vessel and thrombus are omitted). In this case, the elastic area 24 of catheter 20 is solely made of flexible material, including but not limited to NiTi alloy, and elastic polymer such as polyimide, PU, TPU, silicone, rubber . . . etc. Like the spring structure of FIG. 3B, the negative pressure applied by the pump would change the length of the elastic area 24. The operation process is the same as FIGS. 3A to 3B, so no repeat here.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A thrombectomy device comprising:
an aspiration pump for providing an negative pressure continuously or by interval;
a catheter having a distal end, mid portion and a proximal end and defining a longitudinal axis; and
a valve connecting between the aspiration pump and the proximal end of the catheter or connecting to the catheter;
wherein the catheter comprises at least one elastic area, which is compressed along the longitudinal axis in response to application of the negative pressure and expanded along the longitudinal axis in response to relieve of the negative pressure.

2. The thrombectomy device according to claim 1, wherein the distal end of the catheter is the elastic area.

3. The thrombectomy device according to claim 1, wherein the proximal end of the catheter is the elastic area.

4. The thrombectomy device according to claim 1, wherein the mid portion of the catheter is the elastic area.

5. The thrombectomy device according to claim 1, wherein the entire catheter is the elastic area.

6. The thrombectomy device according to claim 1, wherein the elastic area is made of a flexible material.

7. The thrombectomy device according to claim 6, wherein the flexible material includes NiTi alloy and elastic polymer such as polyimide, PU, TPU, silicone, rubber.

8. The thrombectomy device according to claim 1, wherein a spring is disposed in the elastic area of the catheter.

9. The thrombectomy device according to claim 1, wherein the elastic area of the catheter has an accordion fold structure.

10. A method of removing thrombus using the thrombectomy device according to claim 1, comprising following steps:
contacting the distal end of the catheter to a thrombus;
turning on the aspiration pump;
alternately open and close the value to change the length of the elastic area; and
move or break the thrombus with an elastic force generated by the compressed/released elastic area.

11. The method according to claim 10, wherein the distal end of the catheter is the elastic area.

12. The method according to claim 10, wherein the proximal end of the catheter is the elastic area.

13. The method according to claim 10, wherein the mid portion of the catheter is the elastic area.

14. The method according to claim 10, wherein the entire catheter is the elastic area.

15. The method according to claim 10, wherein the elastic area is made of a flexible material.

16. The method according to claim 10, wherein the elastic area of the catheter has an accordion fold structure.

17. The method according to claim 15, wherein the flexible material includes NiTi alloy and elastic polymer such as polyimide, PU, TPU, silicone, rubber.

18. The method according to claim 10, wherein a spring is disposed in the elastic area of the catheter.
